(19) United States
(12) Patent Application Publication
(10) Pub. No.: US 2012/0160176 A1
(43) Pub. Date: Jun. 28, 2012

(54) TREAT DISPENSER AND TREAT DISPENSING SYSTEM

(75) Inventors: Ryan Thomas Murphy, Halifax (CA); Andrew Michael Bruce, Halifax (CA)

(73) Assignee: 24/7 SOFTWARE, Halifax (CA)

(21) Appl. No.: 13/306,597

(22) Filed: Nov. 29, 2011

Related U.S. Application Data
(60) Provisional application No. 61/417,771, filed on Nov. 29, 2010.

(51) Int. Cl. A01K 5/00; A01K 29/00; G06F 17/00

(52) U.S. Cl. 119/57.1; 700/232

(57) ABSTRACT

A treat dispensing system to dispense treats to animals, includes a treat dispenser controlled by a user controller. The treat dispenser includes a housing having an interior and at least one aperture to dispense treats. A treat advancer advances a treat from a storage area to a chute, for example via an actuator, controlled by an advancer controller. The advancer controller is controlled by a treat dispenser controller, which is in turn controlled by the user controller. The user controller may comprise a remote control, cordless telephone, cellular telephone, PDA, network connected desktop or laptop computer. The treat dispenser controller may include a camera and loudspeaker. The user can remotely initiate the advancer to align the treat storage with the chute so that a treat held within the treat storage area is dispensed via the aperture to the external environment.

### CROSS-REFERENCE TO RELATED APPLICATION

This application claims benefit under 37 U.S.C. 119(e) to U.S. patent application Ser. No. 61/417,771, filed Nov. 29, 2010, which is incorporated by reference herein in its entirety.

### BACKGROUND

The present disclosure relates to a treat dispenser and a treat dispensing system.

### BRIEF SUMMARY

A treat dispensing system is operable to selectively dispense treats to animals, such as a domestic pet. The treat dispensing system may be summarized as including a treat dispenser controlled by a user controller. The treat dispenser may include a housing having a hollow interior provided with at least one aperture through which treats are dispensed. Disposed within the housing, is a treat advancer which advances a treat from a storage area to a chute. The treat advancer may be advanced by an advancer or actuator, such as an electric motor. The advancer is controlled by an advancer controller. The advancer controller is controlled by a treat dispenser controller. The treat dispensing controller is in turn controlled by the user controller. The user controller may take a large variety of forms, such as a remote control, cordless telephone, cellular telephone, PDA, network connected desktop or laptop computer, for example. The treat dispenser controller may further comprise a camera and loudspeaker. The camera may capture an image or video for display. The loudspeaker may permit the user to provide sounds to emanate in the vicinity of the treat dispenser. The user can remotely, via the user controller, initiate the advancer such that the treat advancer advances such that the treat storage aligns with the chute so that a treat held within the treat storage area is dispensed via the chute the aperture in the housing to the external environment. The treat may be dispensed using the force of gravity.

### BRIEF DESCRIPTION OF DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of treat dispensing system according to one illustrated embodiment.

FIG. 2 is an exploded view of a treat dispenser according to one illustrated embodiment.

FIG. 3A is a top plan view of the treat dispenser with a cap removed, showing the housing, treat dispensing area and treat storage area, according to one illustrated embodiment.

FIG. 3B is a schematic diagram of treat dispensing system according to one illustrated embodiment.

FIG. 3C is a schematic diagram of treat dispensing system according to one illustrated embodiment.

FIG. 4 is a schematic diagram of the treat dispenser controller according to one illustrated embodiment.

FIG. 5 is a schematic diagram of the user controller according to one illustrated embodiment.

FIG. 6 is a schematic diagram of a method of operating the treat dispensing system, including part of a process to connect user controller to treat dispenser controller according to one illustrated embodiment.

FIG. 7 is a schematic diagram of a method of operating the treat dispensing system including part of the process to connect user controller to treat dispenser controller according to one illustrated embodiment.

FIG. 8 is a schematic diagram of the user controller connected to treat dispenser controller according to one illustrated embodiment.

FIG. 9A is an isometric view of the housing according to one illustrated embodiment.

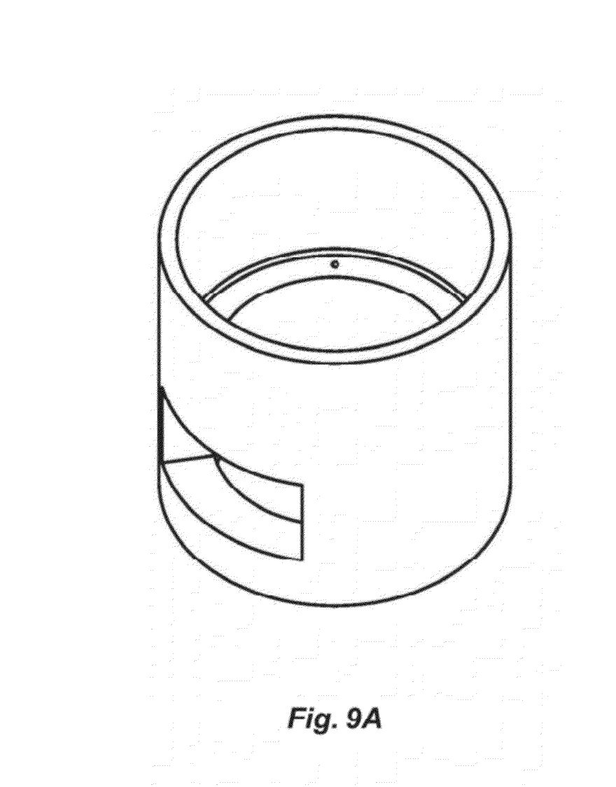

FIG. 9B is a top down view of the housing according to illustrated embodiment.

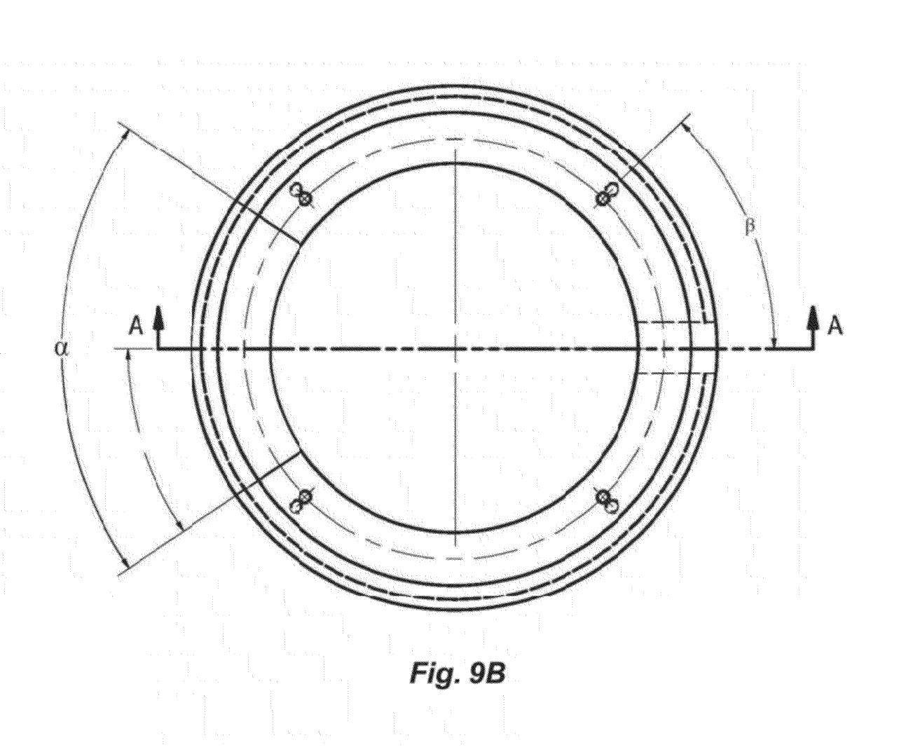

FIG. 9C is a cross sectional view of the housing at line A-A of FIG. 9b, according to the illustrated embodiment.

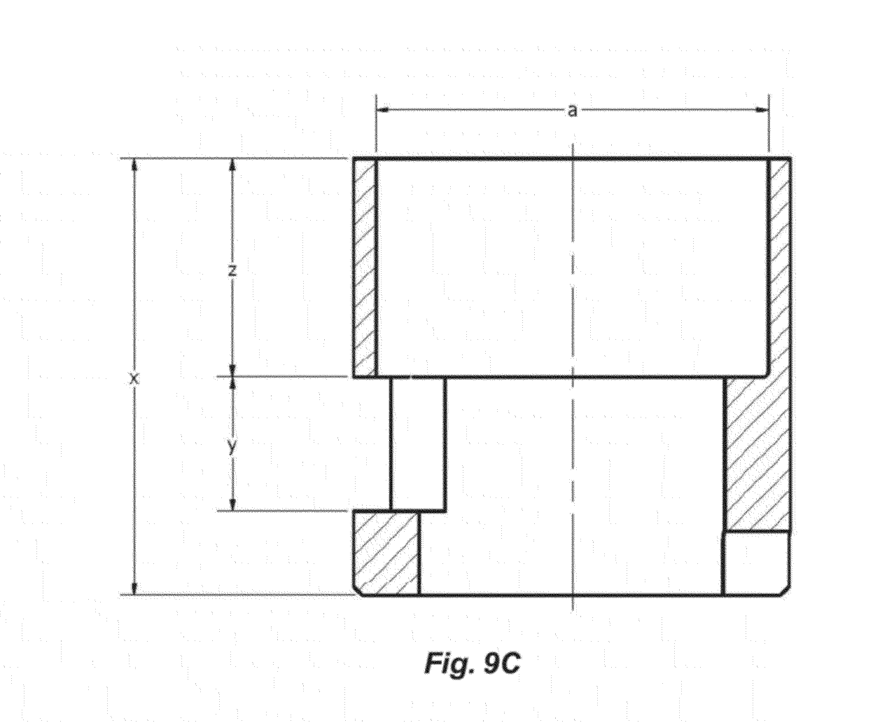

FIG. 9D is a side elevational view of the housing, according to the illustrated embodiment, showing an internal structure of the housing in broken line.

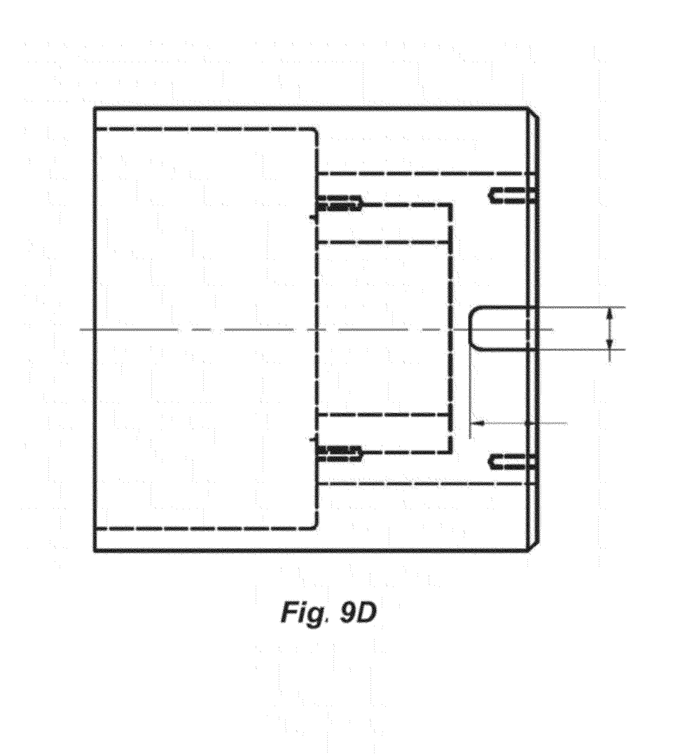

FIG. 9E is a top plan view of the housing according to the illustrated embodiment.

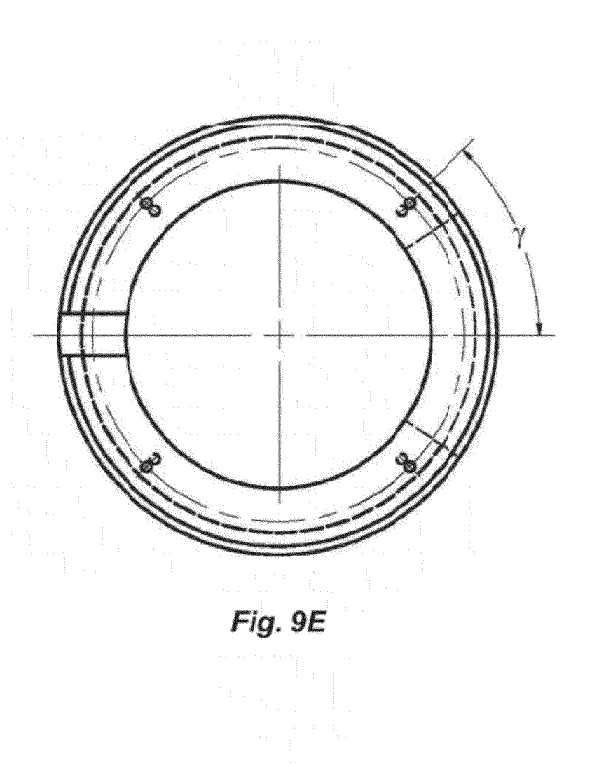

FIG. 10A is a top plan view of a divider according to one illustrated embodiment.

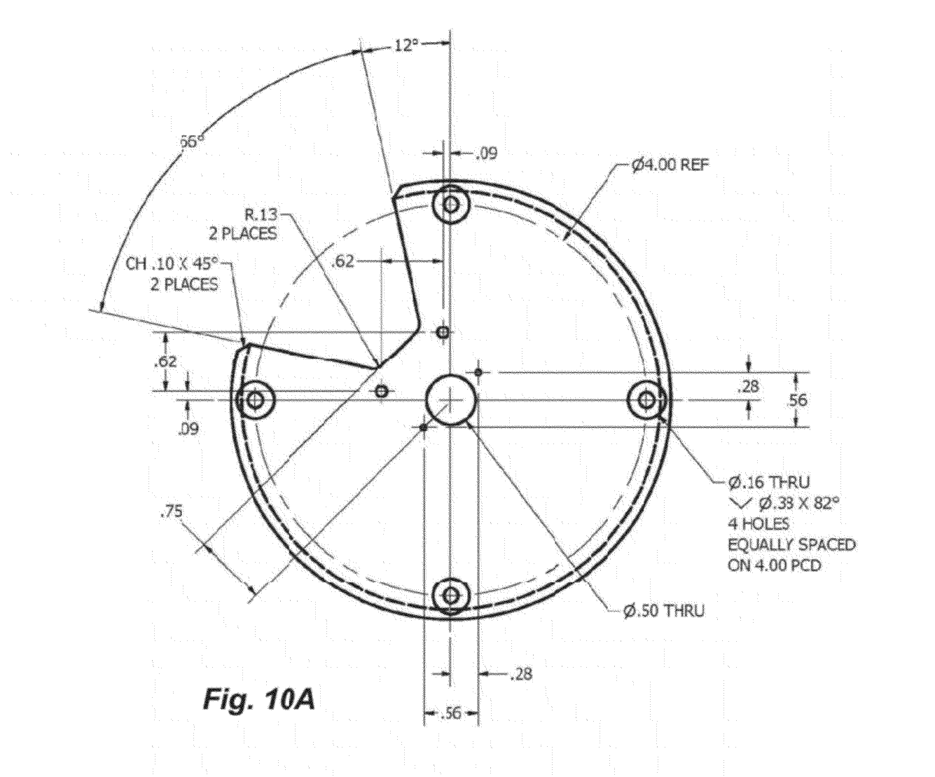

FIG. 10B is a side elevational view of the divider according to the illustrated embodiment.

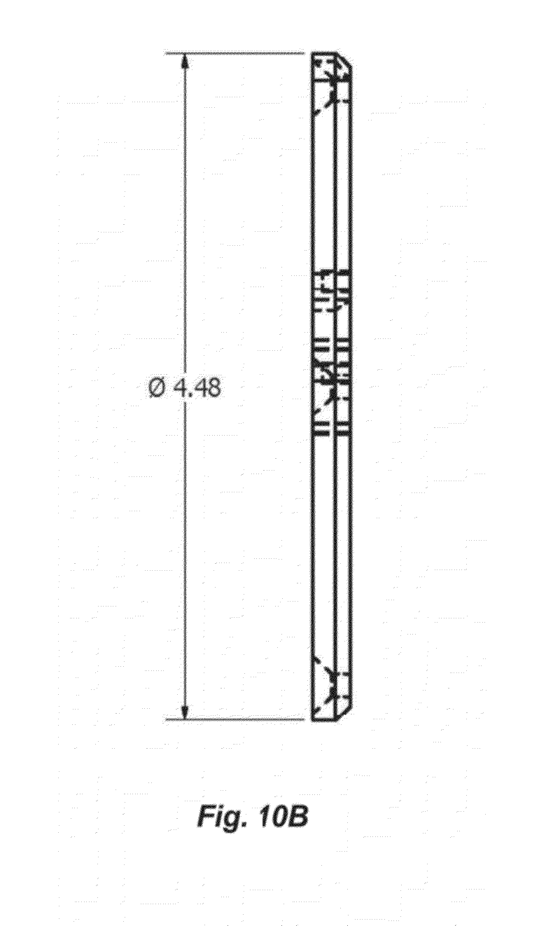

FIG. 10C is an isometric view of the divider according to the illustrated embodiment.

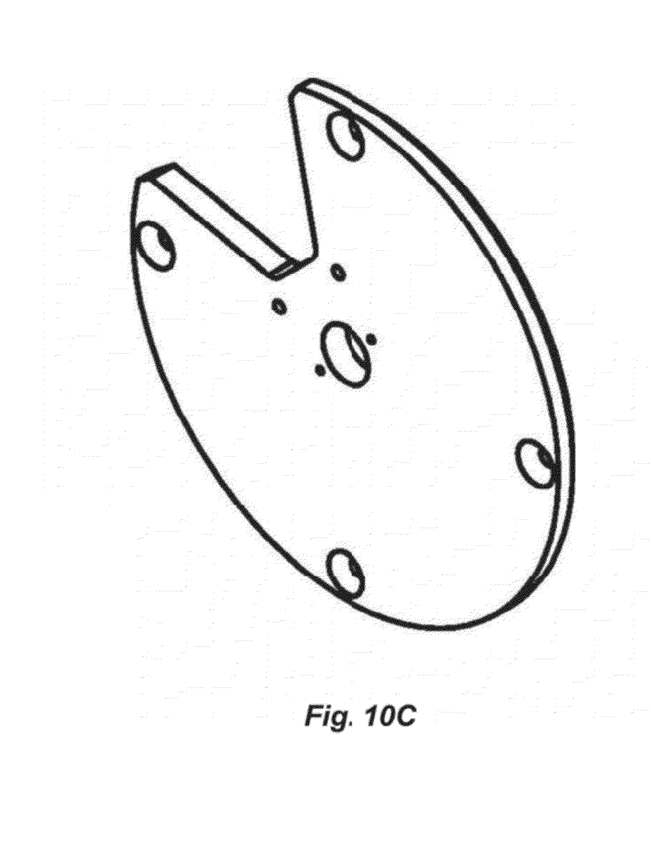

FIG. 11A is an isometric view of a chute according to one illustrated embodiment.

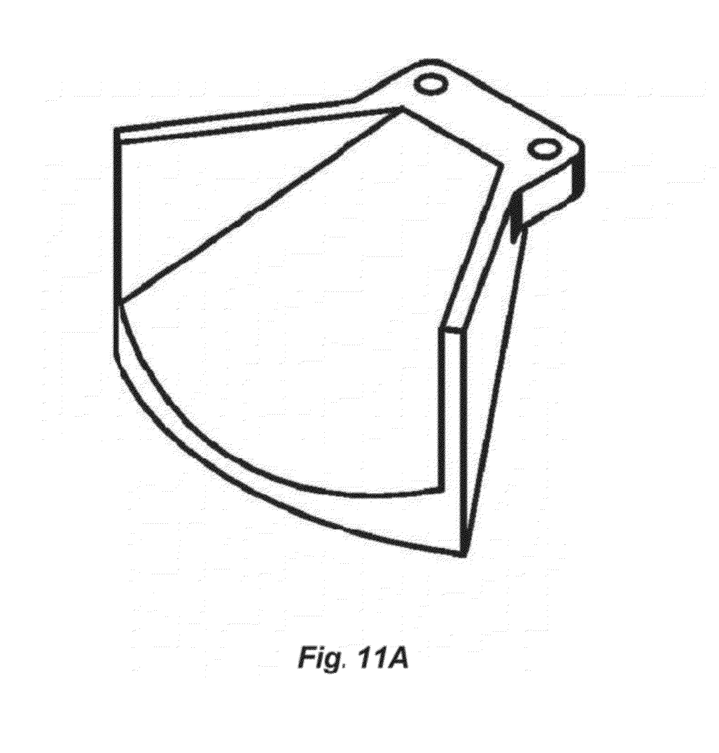

FIG. 11B is a top plan view of the chute according to the illustrated embodiment.

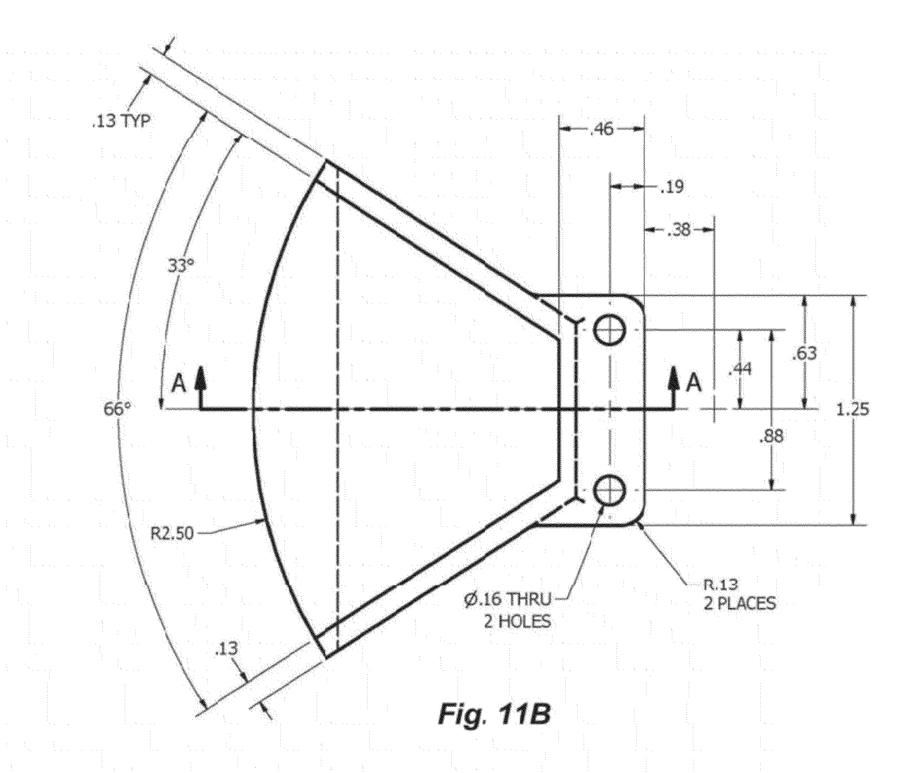

FIG. 11C is a cross sectional view of the chute taken along line A-A of FIG. 11b according to the illustrated embodiment.

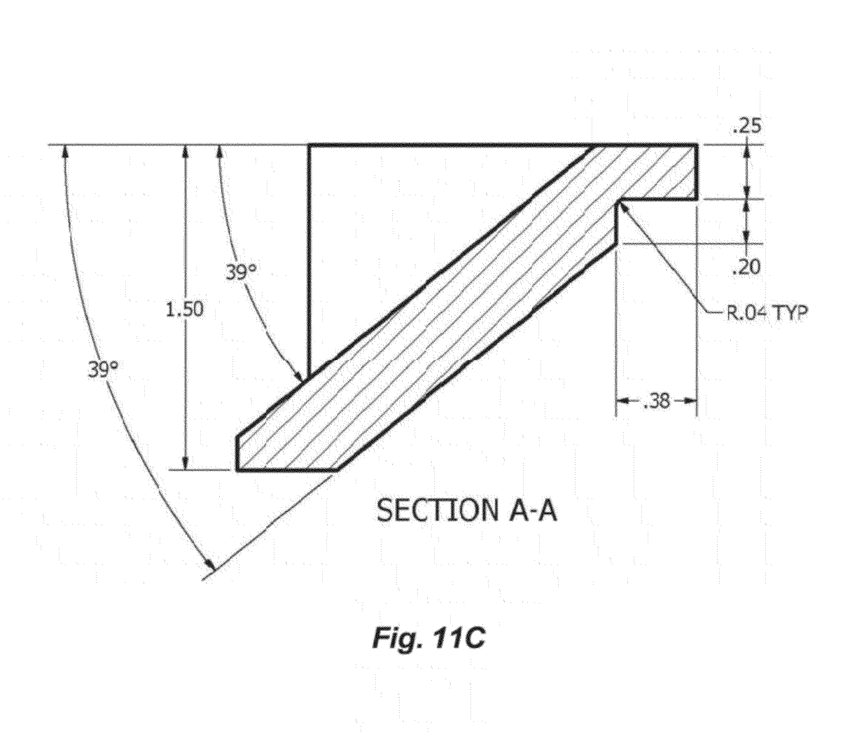

FIG. 12A is a top plan view of a treat advancer according to one illustrated embodiment.

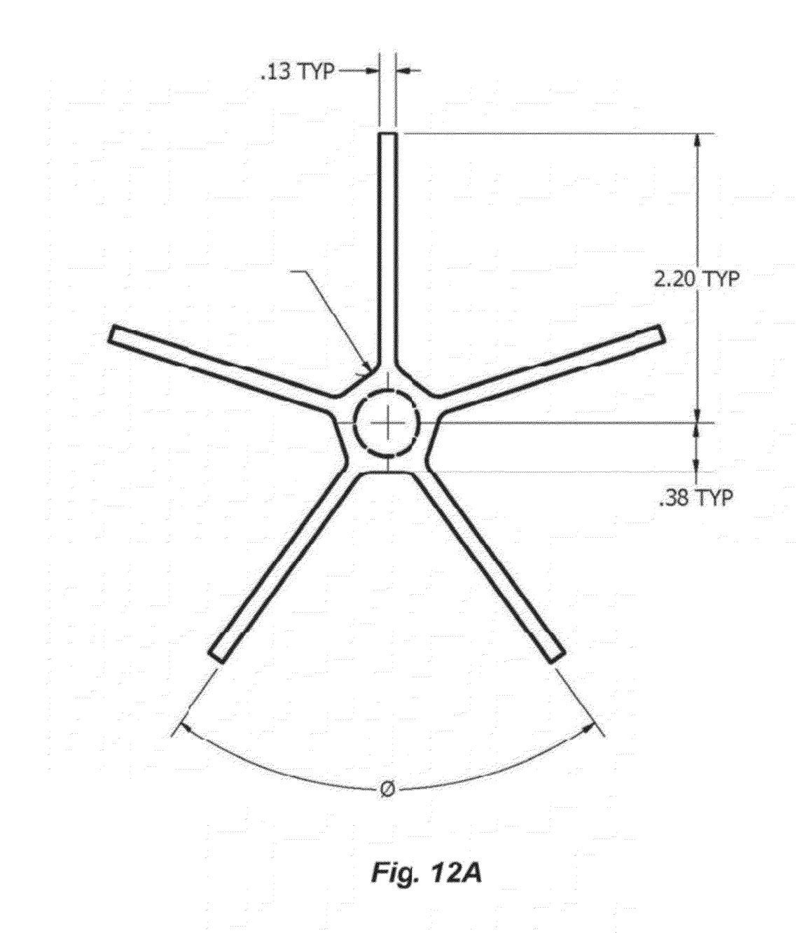

FIG. 12B is a side elevational view of the treat advancer according to the illustrated embodiment.

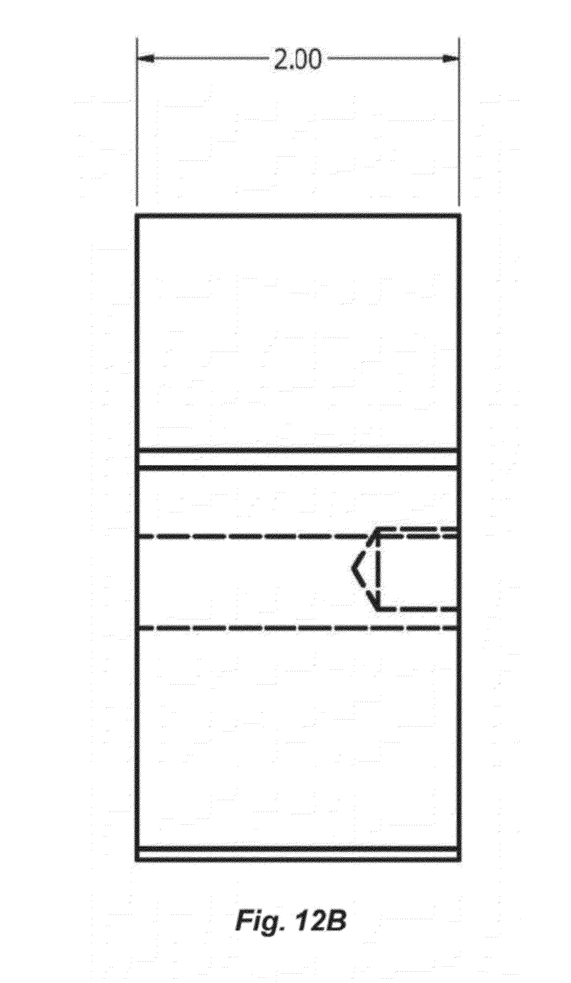

FIG. 12C is a bottom plan view of the treat advancer according to the illustrated embodiment.

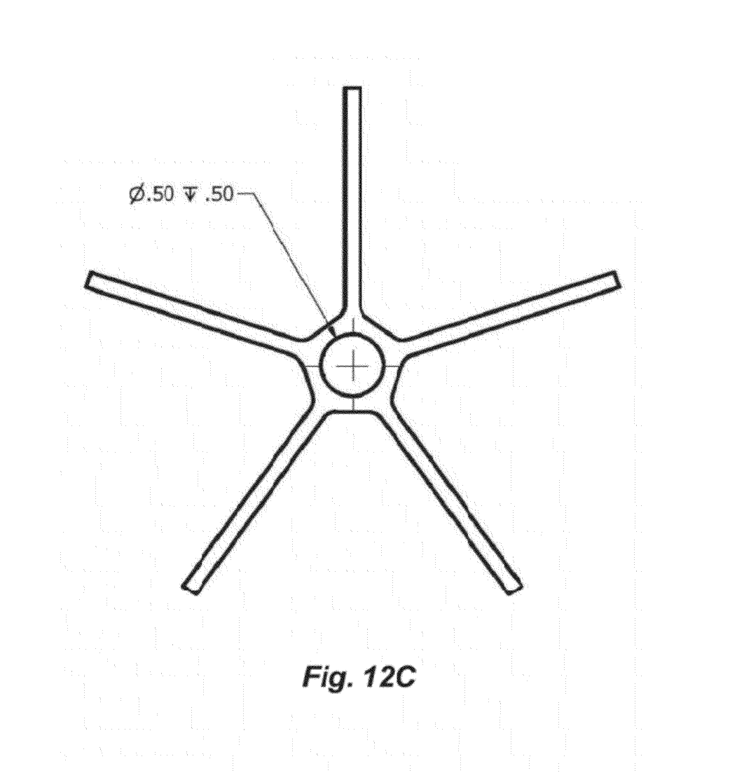

FIG. 12D is an isometric view of the treat advancer according to the illustrated embodiment.

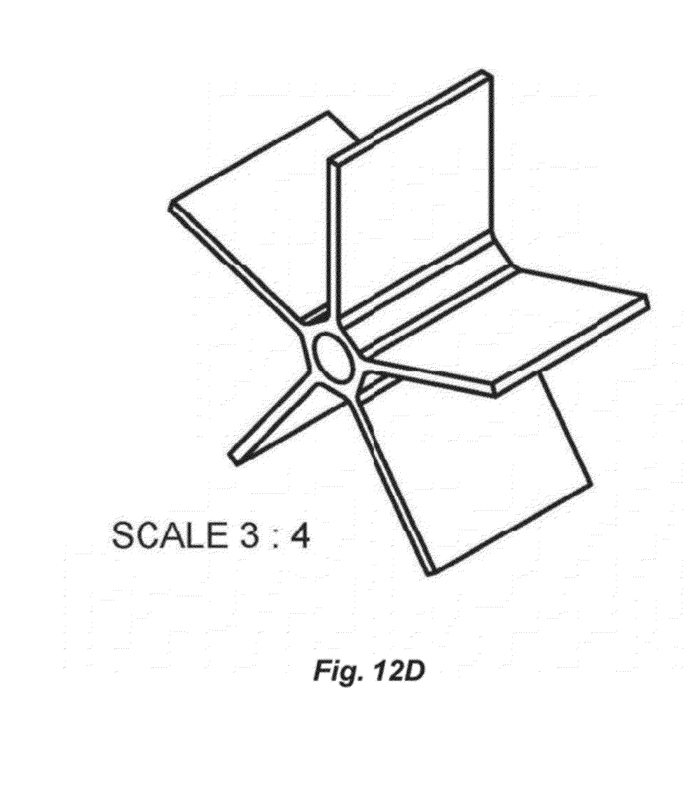

FIG. 13A is a top plan view of a base according to one illustrated embodiment.

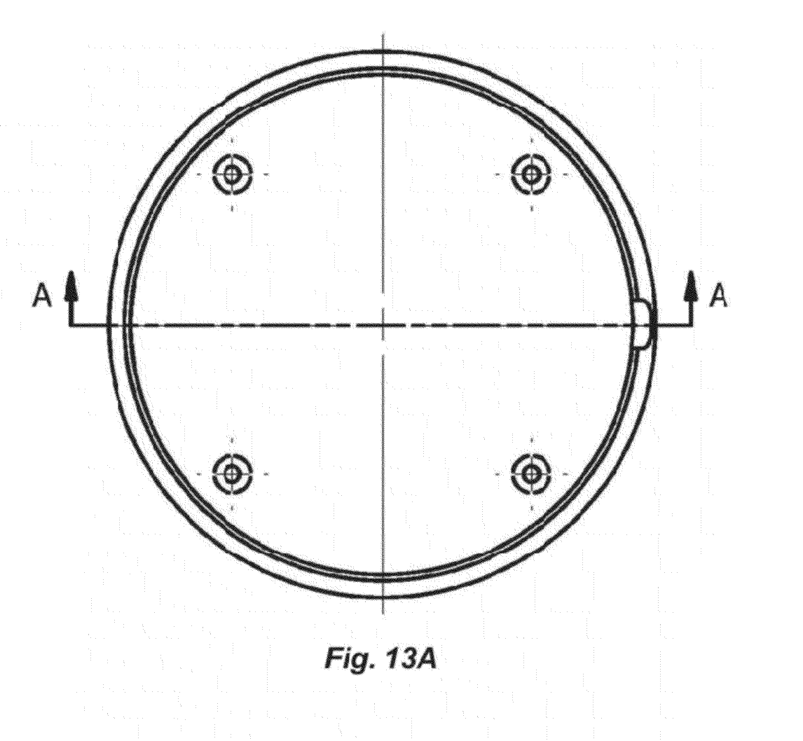

FIG. 13B is an isometric view of the base according to the illustrated embodiment.

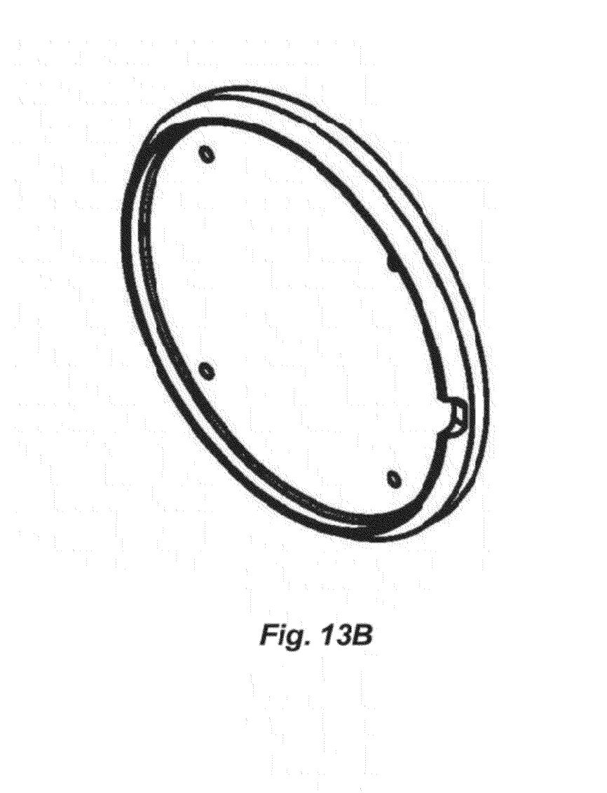

FIG. 13C is a cross sectional view of the base taken along line A-A of FIG. 14a, according to the illustrated embodiment.

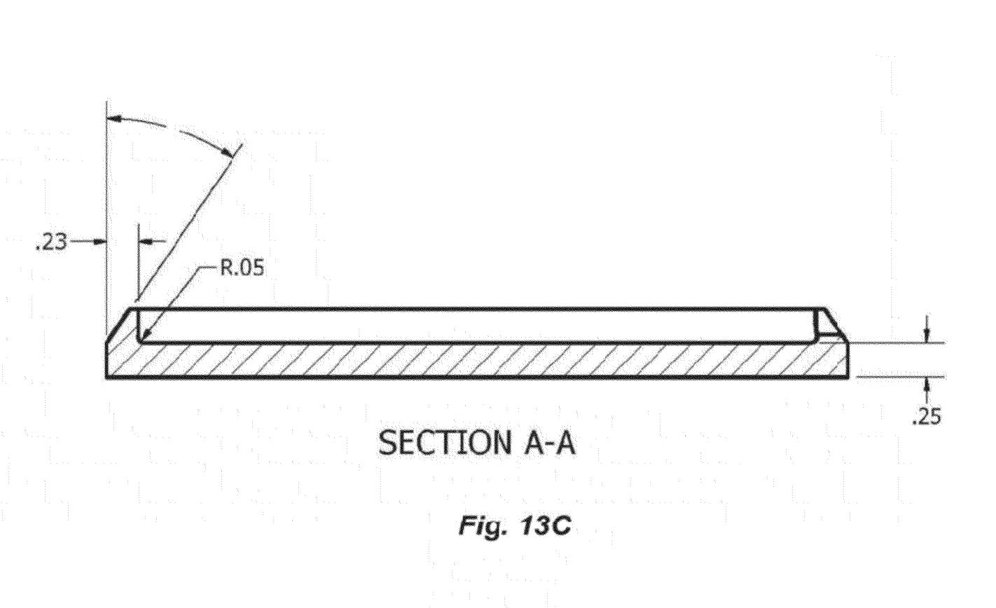

FIG. 13D is a side elevational view of the base according to the illustrated embodiment.

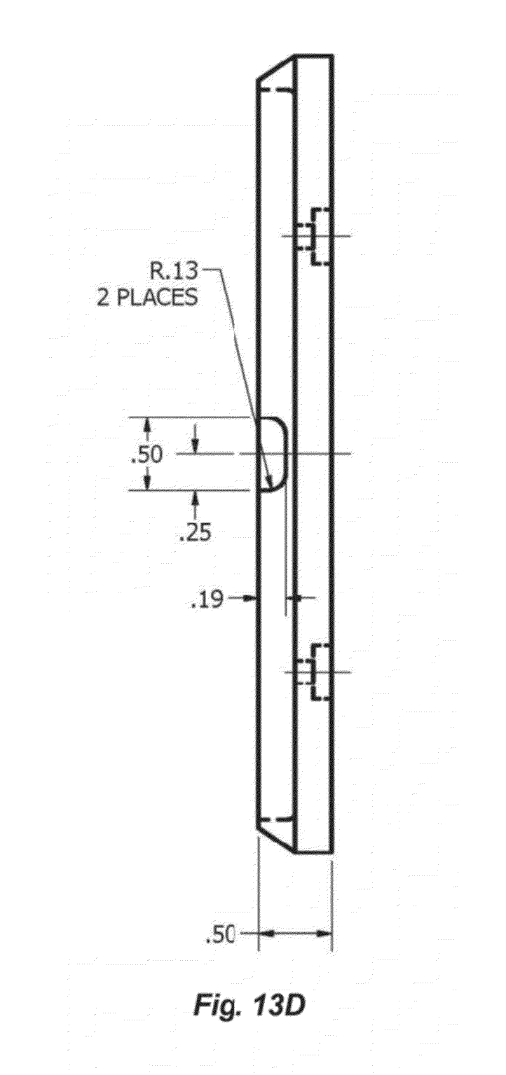

FIG. 13E is a bottom plan view of the base according to the illustrated embodiment.

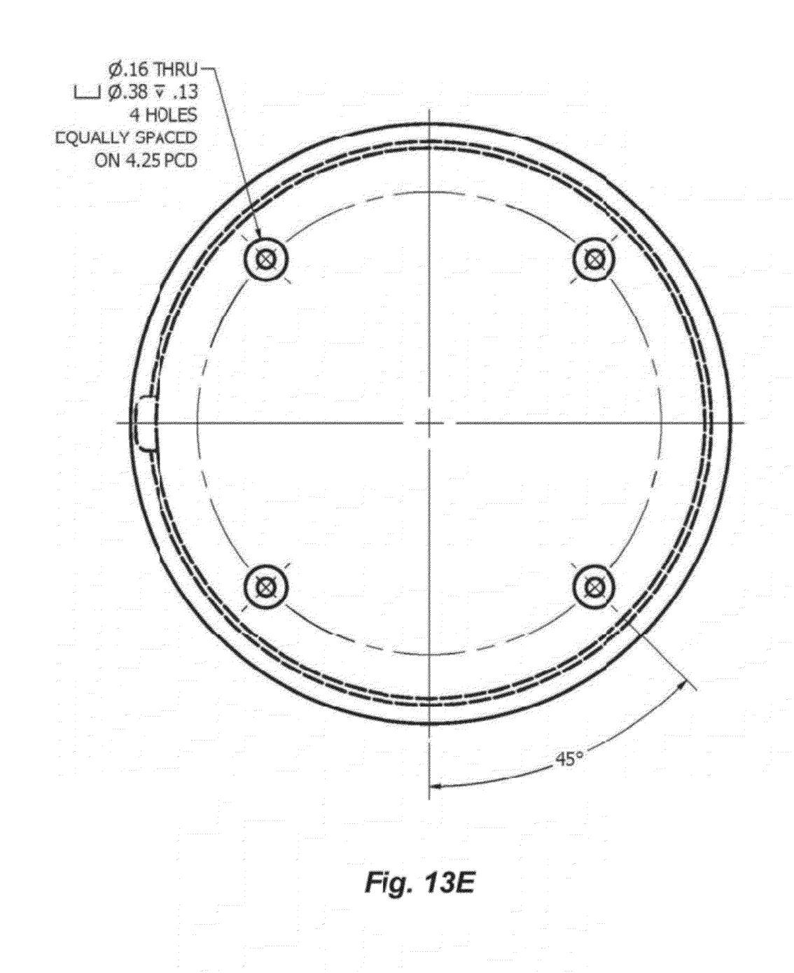

FIG. 14A is a top plan view of a cap according to one illustrated embodiment.

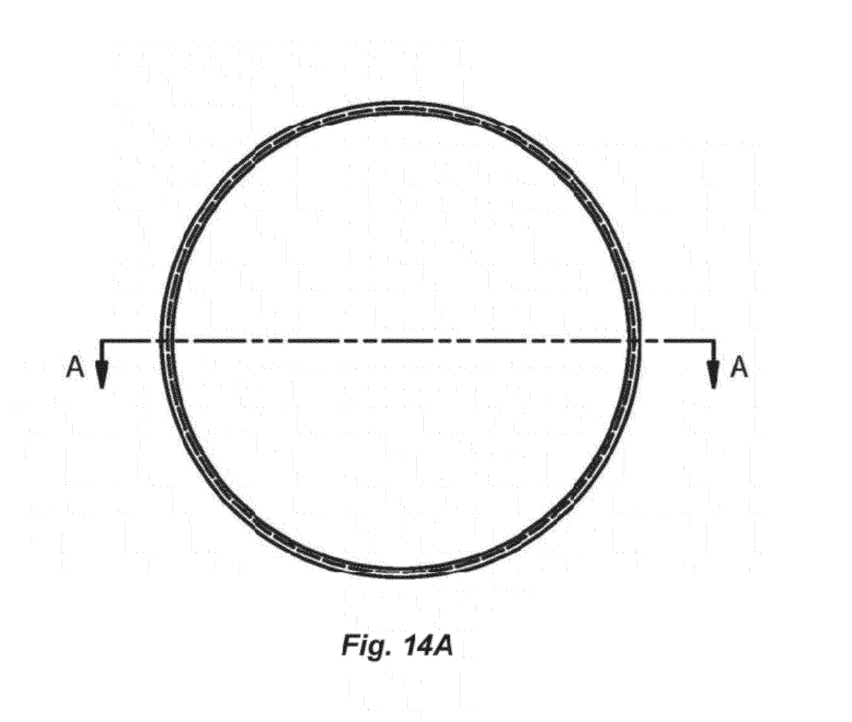

FIG. 14B is a cross sectional view of the cap taken along line A-A of FIG. 15a, according to the illustrated embodiment.

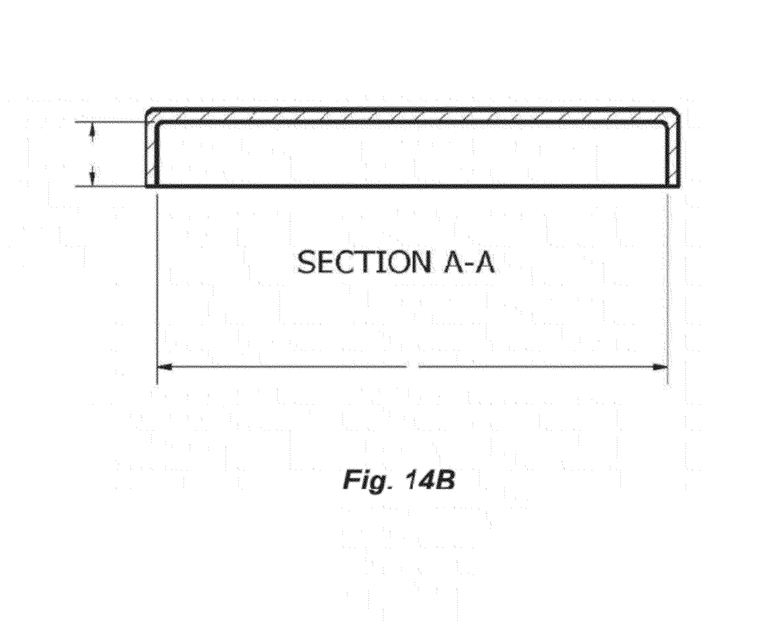

FIG. 14C is an isometric view of the cap according to the illustrated embodiment.

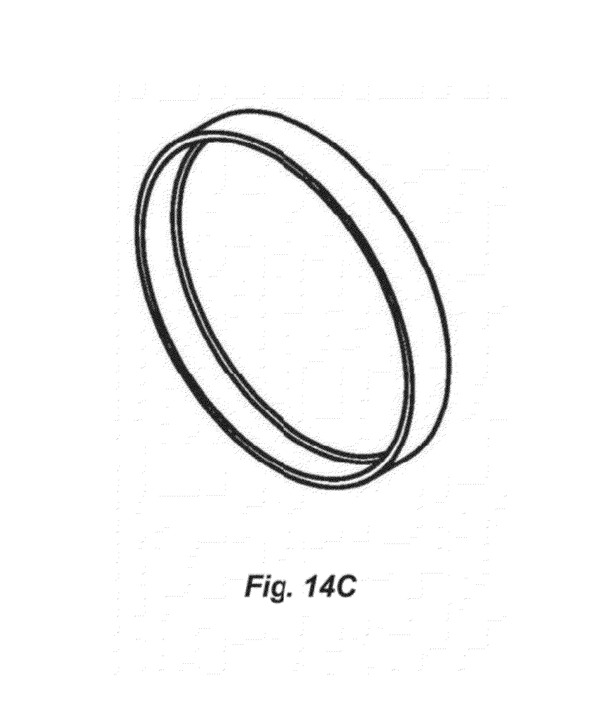

FIG. 15A is an isometric view of a treat feeder according to one illustrated embodiment.

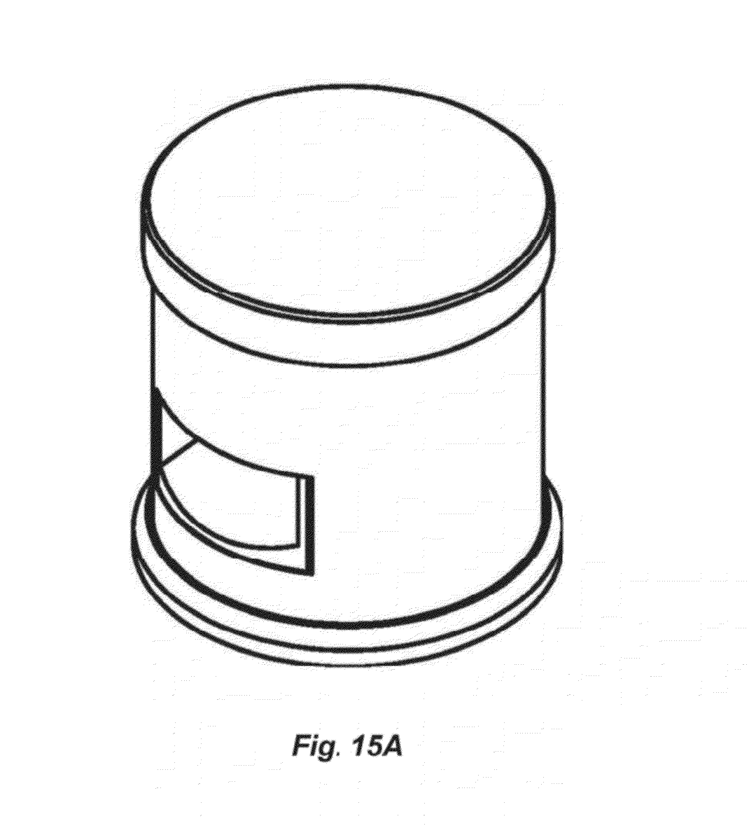

FIG. 15B is a side elevational view of the treat feeder according to the illustrated embodiment.

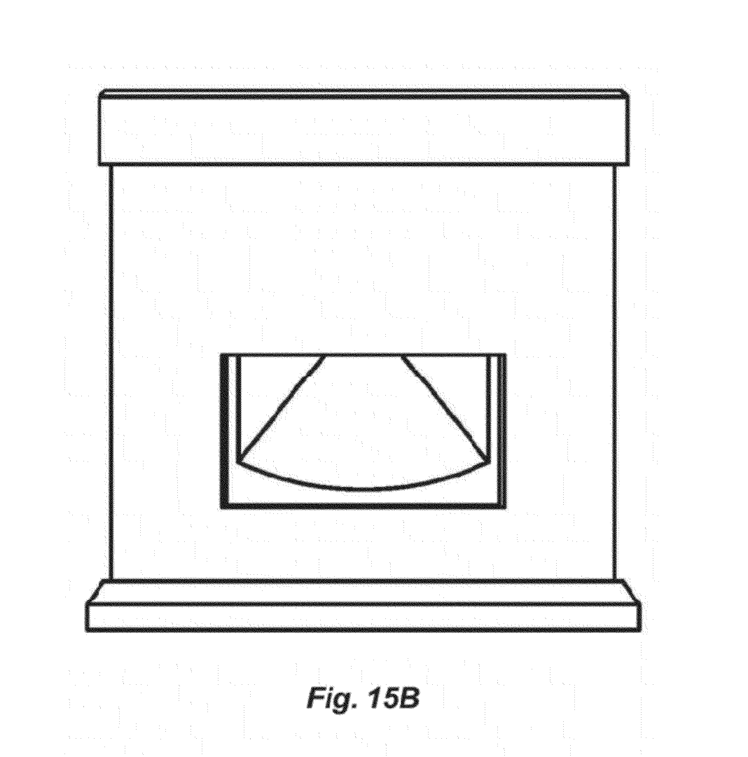

FIG. 15C is a bottom plan view of the treat feeder according to the illustrated embodiment.

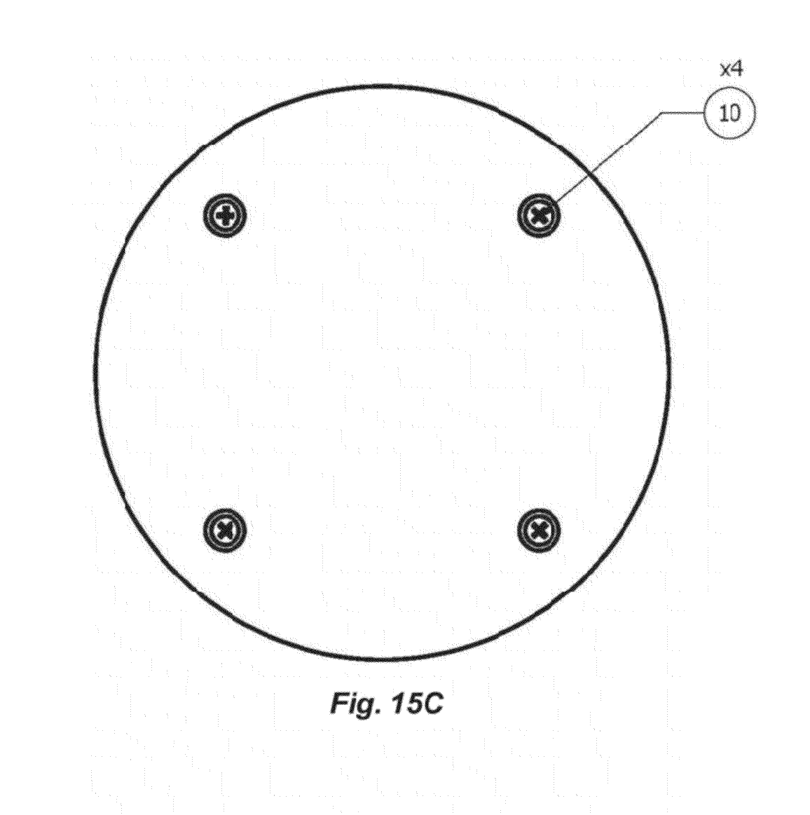

FIG. 16A is a schematic diagram of an electric circuit of the advancer controller according to one illustrated embodiment.

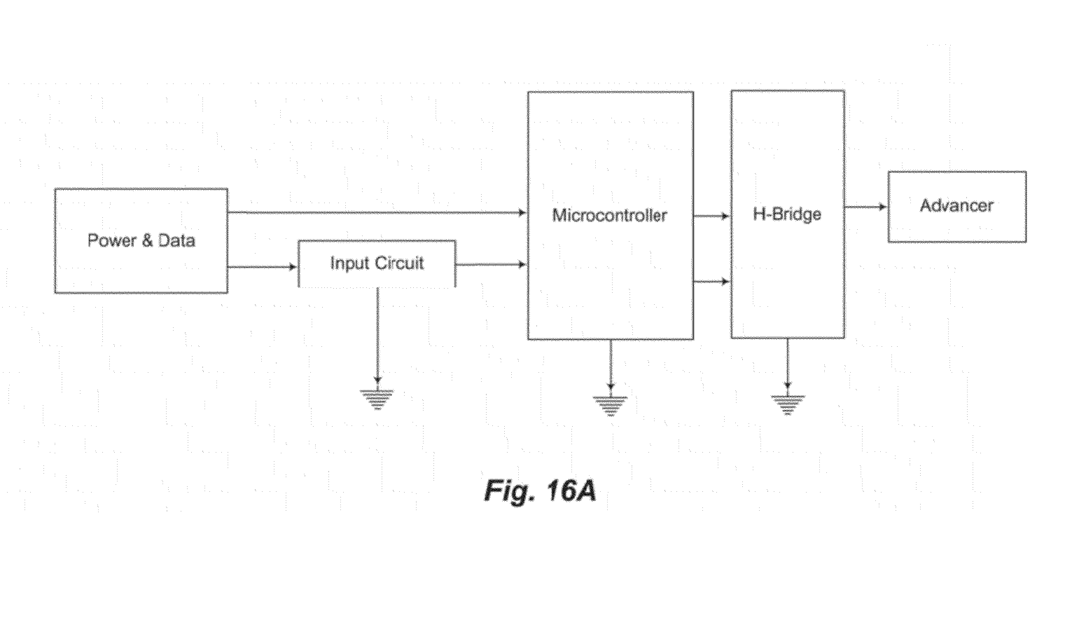

FIG. 16B is a schematic diagram of the electric circuit board of the advancer controller according to one illustrated embodiment;

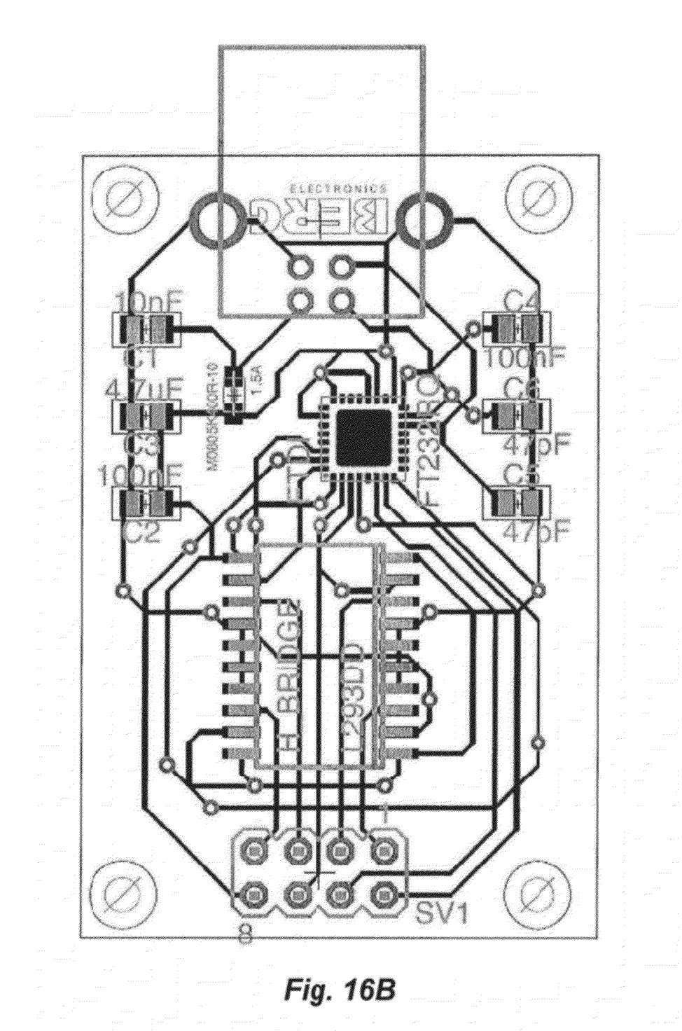

### DESCRIPTION OF SPECIFIC EMBODIMENTS

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with transmitters, receivers, or transceivers and/or dispensers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

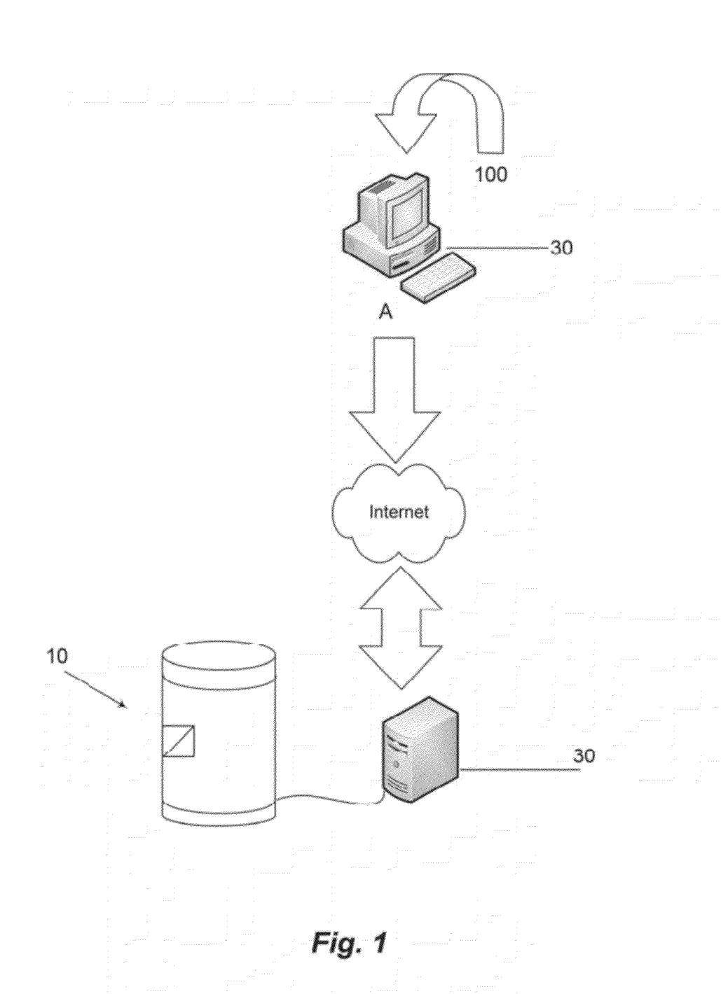

Referring now to the drawings in detail, FIG. 1 a treat dispenser system 100 according to one exemplary illustrated embodiment. Treat dispenser system 100 is comprised of a treat dispenser 10, a treat dispenser controller 20, and a user controller 30. The treat dispenser 10 are communicatively connected to one another via one or more communications channels, for instance a wired (e.g., cable) or a wireless communications (e.g., radio, microwave or optical) channel. The treat dispenser controller 20 and a user controller 30 are communicatively connected to one another via one or more communications channels, for instance a wired (e.g., cable) and/or a wireless communications (e.g., radio, microwave or optical), which may include one or more networks (e.g., Internet, intranet, extranet, local area network or LAN, wide area network or WAN, telecommunications networks such as POTS or cellular networks including GSM or CDMA based cellular network)) employing any variety of network architectures, devices (e.g., servers, routers, bridges) and/or protocols (e.g., TCP/IP).

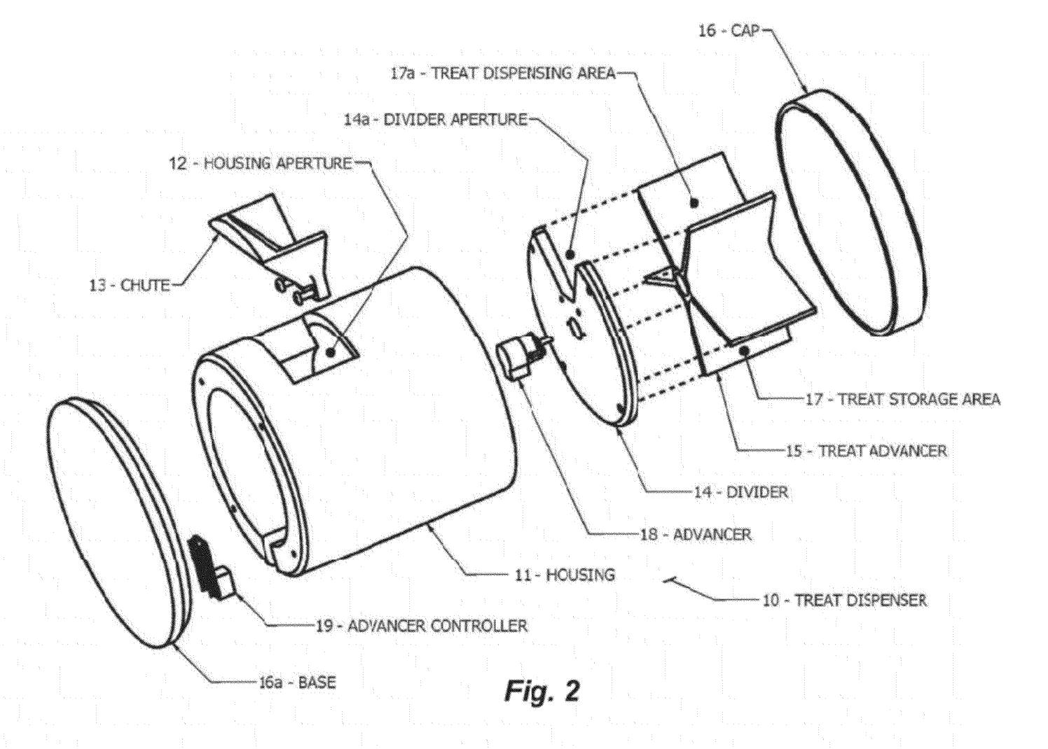

FIG. 2 is an exploded isometric view of the treat dispenser 10. Treat dispenser 10 comprises a housing 11 having with a housing aperture 12. Disposed within the housing aperture 12 is a chute 13. Also disposed within housing 11 is a divider 14. Divider 14 is has a divider aperture 14a. Divider aperture 14a is further aligned with chute 13. Also provided within housing 11 is a treat advancer 15. Housing 11 is enclosed at one end by a cap 16. Housing 11 is enclosed on another end by a base 16a. Base 16a may be a separate element physically attached to the housing 11 or may be a unitary single piece portion of housing 11. Housing 11, divider 14, cap 16 and treat advancer 15 define at least one treat storage area 17 as shown by broken lines in FIG. 2. Treat advancer 15 may be comprised of a paddle wheel, auger or other suitable advancing mechanism. In the embodiment depicted, treat advancer 15 has a number of fins or vanes (e.g., five vanes) so as to define a plurality of storage areas (e.g., four treat storage areas) 17. For clarity of illustration, only one treat storage area 17 is called out in FIG. 2. A further area is defined by the housing 11, divider aperture 14a, cap 16 and treat advancer 15 namely the treat dispensing area 17a as shown by the broken lines in FIG. 2. Treat advancer 15 mechanically, electromechanically, or magnetically coupled with, and is advanced by, an actuator or advancer 18. Actuator or advancer 18 is controlled by an advancer controller 19.

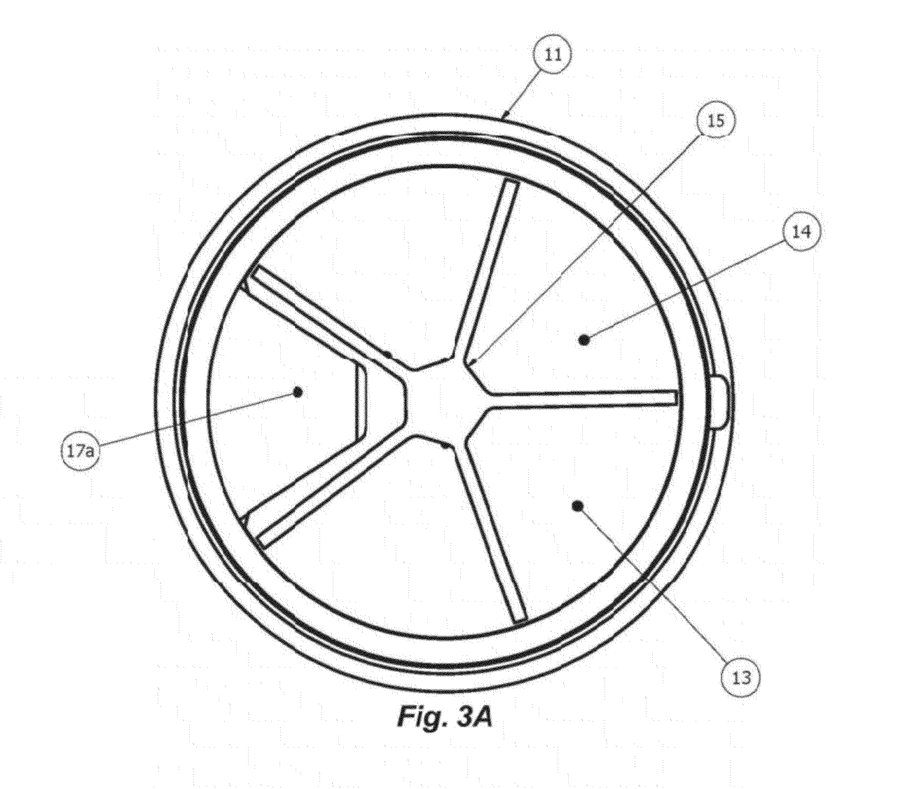

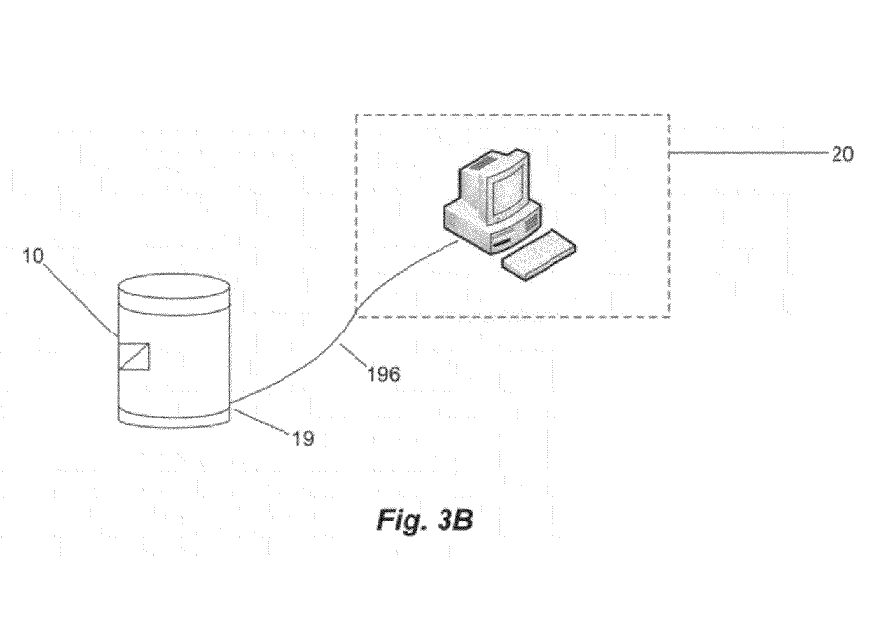

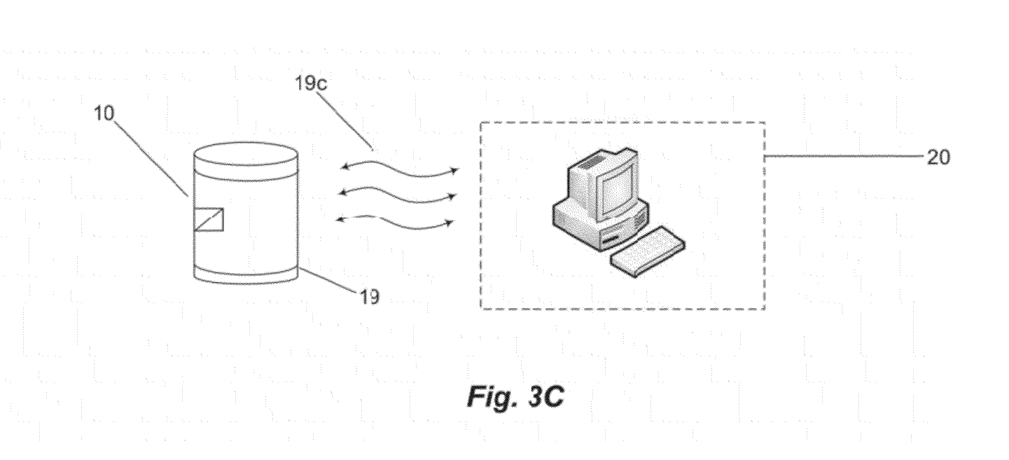

FIG. 3A is a top plan view of the treat dispenser 10 with the cap 16 removed. FIG. 3A shows the housing 11, treat advancer 15 and divider 14. FIG. 3A also shows treat storage area 17 and treat dispensing area 17a.

Housing 11 may be tubular, for example circularly cylindrical or any other suitable shape cross-section or profile (e.g., hexagonal, rectangular). Housing 11 may include a shelf or may have a plurality of internal diameters to provide a surface upon which to mount other elements. Housing aperture 12 may be square, rectangular, circular, ellipsoidal or amorphous in shape provided the housing aperture 12 is sized and shaped to suitably dispense treat(s). Chute 13 may be a separate element attached to the housing 11 or may be integral therewith as a unitary single piece structure with the housing 11. Advancer 18 may be a stepper motor, solenoid, relay or some other electromechanical or magnetic actuator selectively operable to engage and move the treat advancer 18 from a position where the treat storage area 17 aligns with the divider 14 to a position where the treat storage area 17 aligns with the divider aperture 14a and hence becomes a treat dispensing area 17a. The cap 16 and housing 11 may be threaded to permit the cap 16 to be screwed onto the housing 11. Alternatively, the cap 16 may have surfaces sized and dimensioned to frictionally engage complimentary surfaces of the housing 11. Housing 11, chute 13, divider 14, treat advancer 15 and cap 16 may be manufactured out of plastic, metal, wood or any other suitable material. Advancer controller 19 is communicatively coupled to receive commands from the treat dispenser controller 20, and to provide information to treat dispenser controller 20, for example via wired interface, for instance via electrical cable, such as a USB cable or optical cable such as optical fibre, or via wireless interface, for instance via radio transmitter, receiver or transceiver transmitting at radio or microwave frequency, optical transceiver such as infrared optical transceivers or via other electromagnetic radiation.

FIG. 3B shows, according to one embodiment, advancer controller 19 coupled to receive commands and power from treat dispenser controller 20, and to provide information to treat dispenser controller 20 via USB cable 19b,

FIG. 3C shows, according to one embodiment, advancer controller 19 communicatively coupled to receive commands from treat dispenser controller 20, and to provide information to treat dispenser controller 20 via electromagnetic radiation depicted as 19c in FIG. 3C,

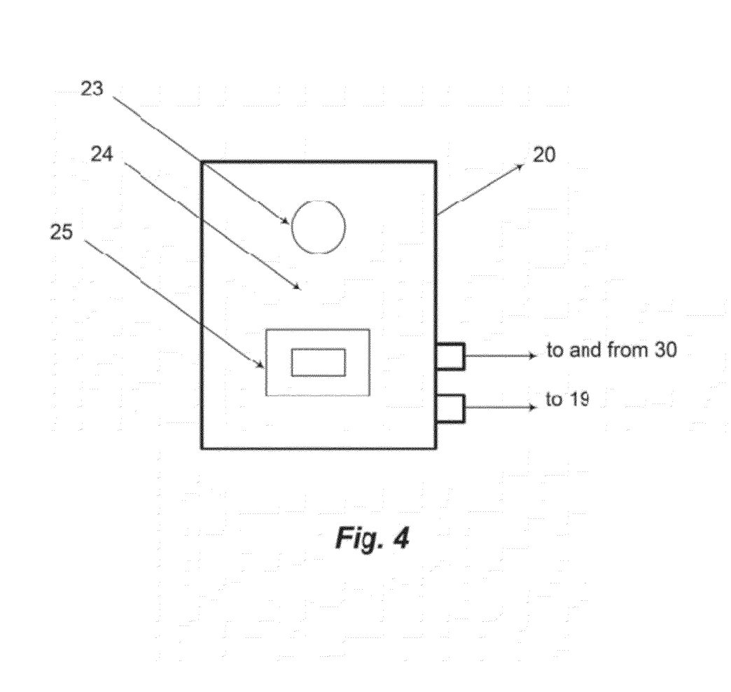

FIG. 4 shows, according to one embodiment, treat dispenser controller 20. Treat dispenser 20 has a camera (e.g., digital camera or image acquisition device) 23, a treat dispenser controller loudspeaker 24 and treat dispenser microphone 25. Treat dispenser controller 20 is communicatively coupled to receive information from the user controller 30 and to provide information to the advancer controller 19. In particular, camera 23 is captures images for transmission to user controller 30. Treat dispenser controller loudspeaker 24 produces sounds based on audio or sound information received from the user controller 30. Treat dispenser microphone 25 is a transducer that transforms sound into audio or sound information (e.g., WAV, AIFF, AU, MPEG formatted files) that is provided to user controller 30, via the treat dispenser controller 20. Advancer controller 19 may be incorporated within treat dispenser controller 20. Treat dispenser controller 20 may take any of a large variety of forms including a desktop computer, laptop computer, dedicated base station, or other similar device.

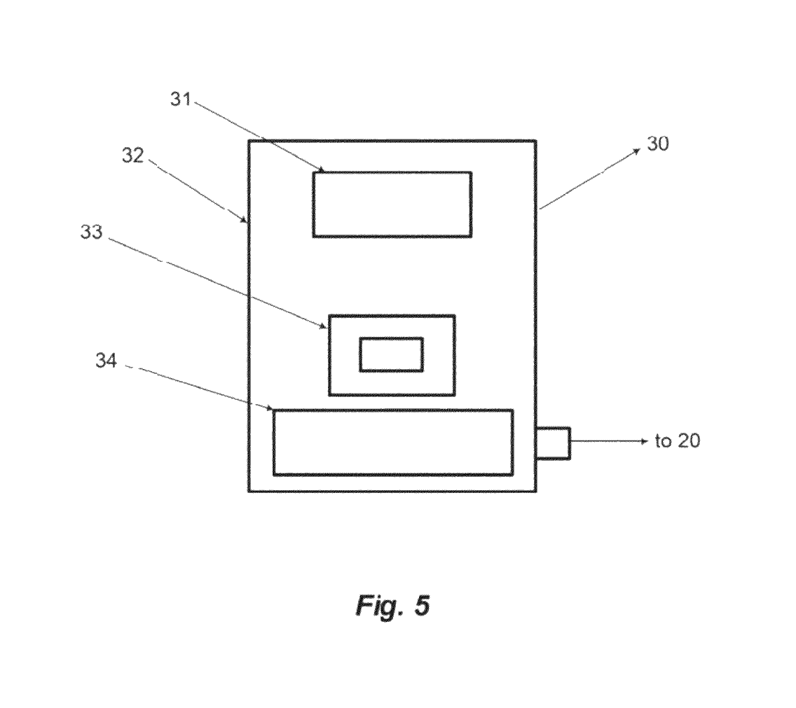

FIG. 5 shows, according to one embodiment, user controller 30. User controller 30 has a display 31, a user controller microphone 32, a user controller loudspeaker 33, and a user controller input 34. User controller 30 is communicatively coupled to provide information from user controller 30 to treat dispenser controller 20. User controller 30 is also communicatively coupled to receive information from treat dispenser controller 20. In particular, display 31 is configured to display images based on information captured and/or received from camera 23. User controller loudspeaker 33 is configured to produce sounds based on audio information or files received from treat dispenser controller microphone 25. User controller microphone 32 is a transducer that transforms sounds from a user into audio or sound information. User controller input 34 is communicatively coupled to receive commands from a user. User controller 20 may take a large variety of forms, for example a desktop computer, laptop computer, net book computer, tablet computing device (e.g., IPAD®), cellular telephone, personal digital assistant, network device, or other similar device.

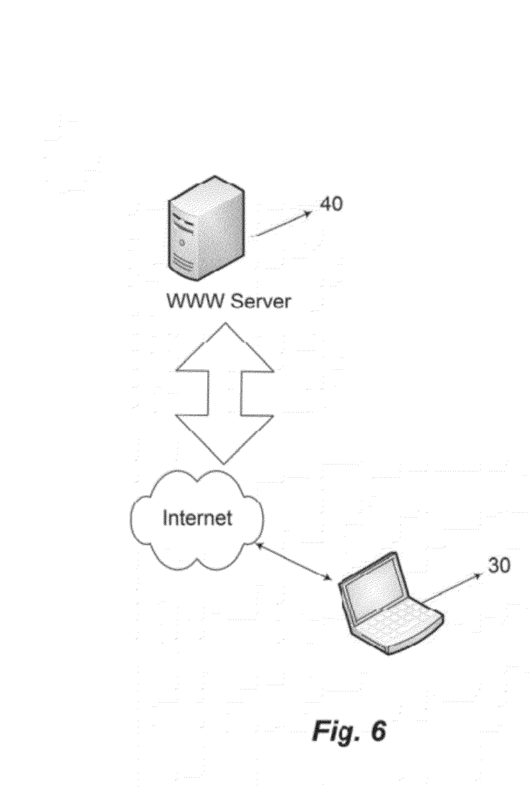

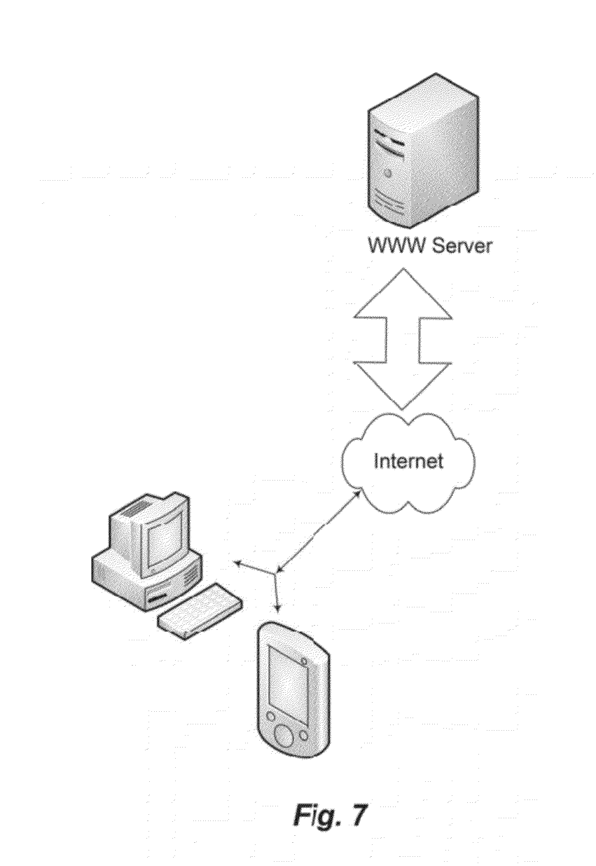

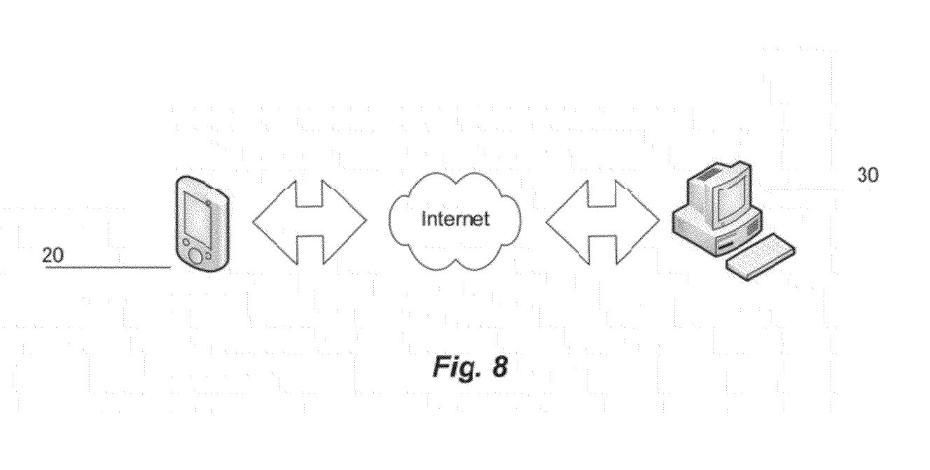

In operation, treat dispenser 10 is loaded with treats by opening the cap 16 and placing a treat into a treat storage area 17. The user may then, from a remote location, initiate a connection between user controller 30 and treat dispenser controller 20. An exemplary process for initiating access between user controller 30 and treat dispenser controller 20 is set out in FIGS. 6, 7 and 8. As shown in FIG. 6, the connection between user controller 30 and treat dispenser controller 20 is initiated by a user running the applicable software on the treat dispenser controller 20. This software allows the treat dispenser controller 20 to communicate with an external server 40. The software is configured to update the IP address of the treat dispenser controller 20 periodically. As shown in FIG. 5, user controller 30 (shown depicted in the embodiment depicted in FIG. 7 as a smartphone) is initiated to communicatively connect to the external server 40 via a Web browser pointed to a specific URL and/or by running a dedicated application software on the user controller 30. The user may login or provide certain authentication credentials. Once authentication credentials are verified by the external server 40, user controller 30 is able to receive and transmit information directly to treat dispenser controller 20 via the internet, as shown in FIG. 8. For example, once the communications connection is made from user controller 30 to treat dispenser controller 20, user controller 30 may receive or request image(s) from camera 23 for display on display 31. Sounds from treat dispenser microphone 25 may further be emanated from user controller loudspeaker 33. User controller 30 may also transmit sounds received from the user controller microphone 32 to be emanated from treat dispenser controller loudspeaker 24. Such sounds may be voice from the user. Pre-programmed or pre-recorded sounds may also be initiated from user controller input 35. User controller input 35 may initiate a command to the treat dispenser controller 20 which would in turn propagate or initiate a command to advancer controller 19 which would in turn propagate or initiate a command to advancer 18 which would then physically move the treat advancer 15 such that the treat storage area 17 is sufficiently aligned with divider aperture 14a so as to become a treat dispensing area 17a and so as to permit a treat to fall under the force of gravity from treat dispensing area 17a though divider aperture 14a and through housing aperture 12 via chute 13.

The treat dispenser controller 20 may be physically separated from treat dispenser 10 and communicate with treat dispenser 10 by information propagated wirelessly by electromagnetic radiation. The treat dispenser controller 20 may be physically separated from treat dispenser 10 other than by communication cable such as a USB cable and communicate with treat dispenser 10 via the USB cable. The treat dispenser 10 may be powered by on board battery or other self-contained power source. The treat dispenser 10 may be powered by a power cable to an electrical outlet, with or with the use of an on board batter. Alternatively, treat dispenser 10 may be powered by the communication cable, such as a USB cable. In such an embodiment, the battery or separate external power cable and an associated power supply circuits may be omitted, saving in cost. The treat dispenser controller 20 may be physically integrated into treat dispenser 10. In another embodiment advancer controller 19 is comprised within treat dispenser controller 20.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other dispensers, not necessarily the exemplary treat dispenser systems generally described above. For example, the various embodiments described above can be combined to provide further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A treat dispensing system, comprising:
treat dispenser comprising a housing having an interior and an aperture that provider a first access between the interior and an exterior of the housing, a cap detachably removable from the housing to selectively provide a second access between the interior and the exterior of the housing, a chute disposed within the aperture of the housing, a divider received in the housing and having a divider aperture aligned with the chute, and a treat advancer moveably disposed within the housing, and wherein the housing, the divider, the cap and the treat advancer form at least one treat storage area;
a treat dispenser controller communicatively connected to the treat dispenser; and
a user controller communicatively coupleable to the treat dispenser controller to remotely operate the treat dispenser, wherein

2. The treat dispensing system of claim 1 wherein the treat dispensing controller is operable remotely via the user controller, to control the treat advancer of the treat dispenser to selectively advance the treat advancer such that the treat advancer selectively aligns a respective one of the at least one treat storage area with the divider aperture of the divider.

3. The treat dispensing system of claim 1 wherein the treat advancer is comprised of a paddle wheel.

4. The treat dispensing system of claim 1 wherein the treat advancer is comprised of an auger.

5. The treat dispensing system of claim 1 wherein the treat dispenser controller further comprises a camera.

6. The treat dispensing system of claim 2 wherein the treat dispenser controller further comprises a loudspeaker.

7. The treat dispensing system of claim 2 wherein the treat dispenser controller further comprises a microphone.

8. The treat dispensing system of claim 1 wherein the treat dispenser is powered from the treat dispenser controller via a Universal Serial Bus connector.

9. The treat dispensing system of claim 1 wherein the treat dispenser is powered by a battery.

10. The treat dispensing system of claim 1 wherein the treat advancer controller is communicatively coupled to the treat dispenser controller to receive commands via a Universal Serial Bus connector.

11. The treat dispensing system of claim 1 wherein the treat advancer controller is communicatively coupled to the treat dispenser controller to receive commands via Bluetooth link.

12. The treat dispensing system of claim 1 wherein the treat advancer controller is communicatively coupled to the treat dispenser controller to wirelessly receive commands via a WI-FI network.

13. The treat dispensing system of claim 1 wherein the treat advancer controller is communicatively coupled to the treat dispenser controller to wirelessly receive commands via a 3G network.

14. The treat dispensing system of claim 1 wherein the treat advancer controller is communicatively coupled to the treat dispenser controller to receive commands via a 4G network.
